(19) United States
(12) Patent Application Publication
(10) Pub. No.: US 2022/0117534 A1
(43) Pub. Date: Apr. 21, 2022

(54) METHODS AND APPARATUS FOR A FRAME SURROUNDING A WEARABLE PATCH

(71) Applicant: Otsuka Pharmaceutical Co., Ltd., Tokyo (JP)

(72) Inventors: Tomi MATTILA, Espoo (FI); Mohammadhossein BEHFAR, Espoo (FI); Colm MC CAFFREY, Helsinki (FI); Kimmo JOKELAINEN, Ii (FI); Samuli YRJÄNÄ, Oulu (FI); Antti TAURIAINEN, Oulu (FI); Markku VALKAMA, Oulu (FI)

(73) Assignee: Otsuka Pharmaceutical Co., Ltd., Tokyo (JP)

(21) Appl. No.: 17/436,544

(22) PCT Filed: Mar. 6, 2020
(86) PCT No.: PCT/JP2020/009803

§ 371 Date: Sep. 3, 2021

Related U.S. Application Data
(60) Provisional application No. 62/815,137, filed on Mar. 7, 2019.

(51) Int. Cl. A61B 5/259

(52) U.S. Cl. CPC A61B 5/259; A61B 2562/164; A61B 2562/0217

(57) ABSTRACT

In some embodiments, a system includes a patch assembly and a frame. The patch assembly includes an adhesive portion. The patch assembly is configured to be coupled to a surface of a user via the adhesive portion. The patch assembly has a first patch configuration and a second patch configuration. The frame defines an opening. The patch assembly is configured to be disposed within the opening. The frame has a first frame configuration in which the frame is coupled to the patch assembly via a group of connectors such that the frame prevents the patch assembly from transitioning between the first patch configuration and the second patch configuration. The frame has a second frame configuration in which the group of connectors are broken and the frame is separated from the patch assembly.

### CROSS-REFERENCE TO RELATED APPLICATIONS

This application claims priority to and the benefit of U.S. Provisional Application No. 62/815,137, filed Mar. 7, 2019, entitled “Methods And Apparatus For a Frame Surrounding a Wearable Patch,” the entire content of which is hereby expressly incorporated by reference for all purposes.

### TECHNICAL FIELD

Some embodiments described herein relate generally to systems, methods, and apparatus for coupling a wearable patch assembly to a surface of a user. For example, some embodiments described herein relate generally to systems, methods, and apparatus for coupling an elastic wearable sensor that is able to accommodate skin de-formation to a user.

### BACKGROUND

Patch assemblies can be attached to a surface of a user for various purposes. For example, patch assemblies including a sensor device can be used to non-invasively measure electrical potential differences (e.g., biosignals) between locations on the skin of a human or animal to diagnose and/or monitor a condition of the human or animal. Sensor devices can also be disposed on the skin of a human or animal and be configured to communicate with implanted or digested devices (e.g., digital medicines).

Patch assemblies may be challenging to place properly on the skin of a user due to their tendency to change shape (e.g., twist or wrinkle). Additionally, if an underside of the patch assembly is touched (e.g., by a finger) prior to being placed on the skin, adhesive and/or hydrogel on the underside of the patch assembly may be disrupted.

Thus, there is a need for systems, methods, and apparatus for properly applying patch assemblies to the skin.

### SUMMARY

In some embodiments, a system includes a patch assembly and a frame. The patch assembly includes an adhesive portion. The patch assembly is configured to be coupled to a surface of a user via the adhesive portion. The patch assembly has a first patch configuration and a second patch configuration. The frame defines an opening. The patch assembly is configured to be disposed within the opening. The frame has a first frame configuration in which the frame is coupled to the patch assembly via a group of connectors such that the frame prevents the patch assembly from transitioning between the first patch configuration and the second patch configuration. The frame has a second frame configuration in which the group of connectors are broken and the frame is separated from the patch assembly.

### BRIEF DESCRIPTION OF DRAWINGS

FIG. 1 is a schematic illustration of a system, according to an embodiment.

FIG. 2 is a schematic illustration of a cross-section of a system, according to an embodiment.

FIG. 3 is an illustration of a top view of a patch assembly, according to an embodiment.

FIG. 4 is a perspective exploded view of a patch assembly, according to an embodiment.

FIG. 5 is a perspective exploded view of a system, according to an embodiment.

FIG. 6 is a perspective exploded view of a system, according to an embodiment.

FIG. 7A is an illustration of a system disposed on a surface of a user, according to an embodiment.

FIG. 7B is an illustration of a patch assembly of the system of FIG. 7A disposed on a surface of a user, according to an embodiment.

FIG. 8 is a top view of a system, according to an embodiment.

FIG. 9 is a top view of a system, according to an embodiment.

FIG. 10 is an illustration of a system coupled to a surface of a user, according to an embodiment.

FIG. 11A is a photograph illustrating the steps of using a system to couple a patch assembly to a patient, according to an embodiment.

FIG. 11B is a photograph illustrating the steps of using a system to couple a patch assembly to a patient, according to an embodiment.

FIG. 11C is a photograph illustrating the steps of using a system to couple a patch assembly to a patient, according to an embodiment.

FIG. 11D is a photograph illustrating the steps of using a system to couple a patch assembly to a patient, according to an embodiment.

FIG. 11E is a photograph illustrating the steps of using a system to couple a patch assembly to a patient, according to an embodiment.

FIG. 12 is a flow chart illustrating a method of using a system, according to an embodiment.

FIG. 13 is a top view of a system, according to an embodiment.

FIG. 14 is a close-up view of a portion of the system of FIG. 13.

FIG. 15 is a top view of a system, according to an embodiment.

### DESCRIPTION OF EMBODIMENTS

In some embodiments, a system includes a patch assembly and a frame. The patch assembly includes an adhesive portion. The patch assembly is configured to be coupled to a surface of a user via the adhesive portion. The patch assembly has a first patch configuration and a second patch configuration. The frame defines an opening. The patch assembly is configured to be disposed within the opening. The frame has a first frame configuration in which the frame is coupled to the patch assembly via a group of connectors such that the frame prevents the patch assembly from transitioning between the first patch configuration and the second patch configuration. The frame has a second frame configuration in which the group of connectors are broken and the frame is separated from the patch assembly.

In some embodiments, a system includes a patch assembly, a frame assembly, and a protective layer. The patch assembly includes an adhesive portion configured to couple the patch assembly to a surface of a user. The frame assembly defines an opening. The frame assembly includes a top layer, an adhesive layer, and a liner layer. The adhesive layer is disposed between the top layer and the liner layer. The patch assembly is configured to be disposed within the opening and coupled to the frame via a group of connectors extending between the frame and the patch, and the frame is configured to be removed from the patch assembly by breaking each connector from the group of connectors in a sequential manner such that the connectors are broken in series. The protective layer is coupled to the adhesive portion and disposed in contact with a bottom surface of the liner layer.

In some embodiments, a system includes a patch assembly and a frame. The patch assembly includes an adhesive portion configured to couple the patch assembly to a surface of a user. The patch assembly has an outer perimeter. The frame defines an opening. The patch assembly is configured to be disposed within the opening and coupled to the frame via a group of connectors extending between the frame and the outer perimeter of the patch. A larger portion of the outer perimeter of the patch is free from the group of connectors than a total portion of the outer perimeter of the patch coupled to a connector from the group of connectors. The protective layer is coupled to the adhesive portion and in direct contact with a bottom surface of the frame.

In some embodiments, a method includes separating a portion of a protective layer from a bottom surface of a patch assembly such that the protective layer is decoupled from an adhesive portion of the patch assembly. The patch assembly is disposed within an opening defined by a frame, the patch assembly coupled to the frame via a group of connectors extending between the frame and the patch. The patch assembly and the frame can be disposed on a surface of a user such that the adhesive portion couples the patch assembly to the surface. Each of the connectors can be decoupled from the group of connectors such that the patch assembly remains coupled to the surface and the frame is removed from the surface.

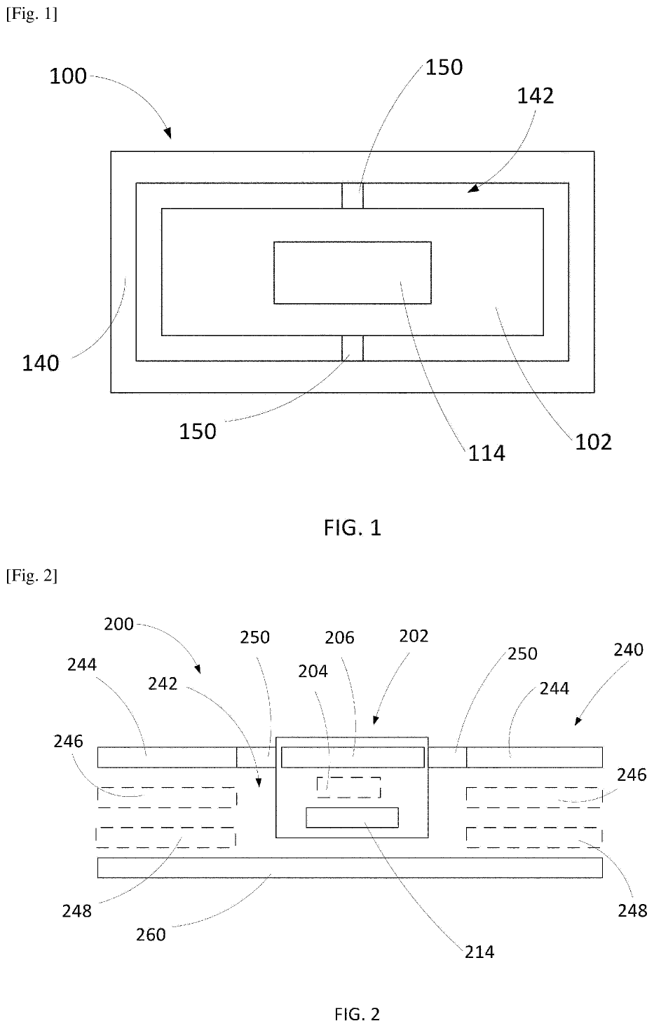

FIG. 1 is a schematic illustration of a system 100. The system 100 includes a patch assembly 102 and a frame 140. The frame 140 defines an opening 142. The patch assembly 102 is disposed within the opening 142. The patch assembly 102 includes an adhesive portion 114. The patch assembly 102 is configured to be coupled to a surface of a user via the adhesive portion 114. The frame 140 has a first frame configuration in which the frame 140 is coupled to the patch assembly 102 via a group of connectors 150 and a second frame configuration in which the group of connectors 150 are broken and the frame 140 is separated from the patch assembly 102. The group of connectors can break such that all or a portion of each connector remains attached to the frame 140 and/or the patch assembly 102 when the frame 140 is in the second frame configuration.

The patch assembly 102 can have a first patch configuration and a second patch configuration. For example, the patch assembly 102 or a portion of the patch assembly 102 may have a different shape (e.g., outer profile) and/or a different length in the first patch configuration compared to the second patch configuration. When the frame 140 is in the first frame configuration relative to the patch assembly 102, the frame 140 can maintain the patch assembly 102 in the first patch configuration via the connectors 150. In some embodiments, the patch assembly 102 or a portion of the patch assembly 102 can be elastic and/or flexible. In some embodiments, the patch assembly 102 can be biased toward the first patch configuration. The frame 140 can be substantially inelastic. For example, the frame 140 can be inelastic along its longitudinal axis such that, when in the first frame configuration when the frame 140 is coupled to the patch assembly 102, the frame 140 can prevent the patch assembly 102 from changing shape via the connectors 150. For example, the frame 140 can prevent the patch assembly 102 from transitioning between the first patch configuration and the second patch configuration.

In some embodiments, the shape of the opening 142 of the frame 140 can correspond to the shape of the patch assembly 102 or a portion of the patch assembly 102. For example, the patch assembly 102 can include a connecting member joining a first portion of the patch assembly 102 and a second portion of the patch assembly 102. The connecting member can have a first configuration when the patch assembly 102 is in the first patch configuration and a second configuration when the patch assembly 102 is in the second patch configuration. The connecting member can include a first segment coupled to a second segment via a flexible hinge. The first segment and the second segment can be arranged at a first angle when the connecting member is in the first configuration. The opening 142 of the frame 140 can include a first opening portion configured to receive the first segment and a second opening portion configured to receive the second segment. The first opening portion can be disposed at a second angle relative to the second opening portion and the second angle can be the same as the first angle. In some embodiments, a connecting member of the patch assembly 102 can have a first sinusoidal shape having a first frequency in the first configuration and a second sinusoidal shape having a second frequency in the second configuration. The second frequency can be different from the first frequency. The opening 142 of the frame 140 can include an opening portion having a sinusoidal shape having the first frequency. In some embodiments, the connecting member can be biased toward the first configuration of the connecting member.

Although the system 100 is shown as having two connectors 150 in FIG. 1, in some embodiments, the system 100 can include any suitable number of connectors 150 (e.g., three, four, five, six, seven, eight, nine, ten or more connectors) arranged in any suitable arrangement. In some embodiments, the connectors 150 can have any suitable size or shape such that the connectors 150 can be broken via applying a force to the frame 140 (e.g., by pulling on the frame 140) such that the frame 140 is separated from the patch assembly 102. For example, the connectors 150 can be shaped as rectangular or triangular segments. The connectors 150 can have a first end coupled to the frame 140 and a second end coupled to the patch assembly 102 and can be tapered from the frame 140 to the patch assembly 102 or from the patch assembly 102 to the frame 140. In some embodiments, a larger portion of an outer perimeter of the patch assembly 102 is free from the connectors 150 than a total portion of the outer perimeter of the patch assembly 102 coupled to a connector 150 from the plurality of connectors 150. In some embodiments, rather than including a number of discrete connectors 150, an interface between the frame 140 and the patch assembly 102 can include perforations such that the frame 140 and the patch assembly 102 can be separated via breaking the perforations. In some embodiments, each connector 150 can be shaped and sized such that the force used to break each connector 150 or a number of connectors 150 simultaneously is less than the force on the patch assembly 102 that would separate the patch assembly 102 from a surface of a user to which the patch assembly 102 is coupled via the adhesive portion 114.

In some embodiments, a larger number of connectors 150 can be coupled to a first portion (e.g., a half) of the patch assembly 102 compared to a second portion of the patch assembly 102 (e.g., the other half). For example, fewer connectors 150 can be coupled to a first half of the patch assembly 102 compared to a second half of the patch assembly 102. The first half of the patch assembly 102 can be a side of the patch assembly 102 in which the separation between the patch assembly 102 and the frame 140 is intended to be initiated (e.g., via pulling the frame 140 away from the patch assembly 102 to break the connectors 150). In some embodiments, a portion of each connector 150 can be formed of one or more different materials than a remainder of each connector 150 such that each connector 150 breaks in a particular location (e.g., the particular location of each connector 150 requires reduced shear forces to break compared to a remainder of each connector 150). In some embodiments, each connector 150 or a portion of each connector 150 can be formed of a different material than one or more layers of the frame 140 (e.g., a top layer of the frame) and/or the patch assembly 102 (e.g., a top layer of the patch assembly 102).

In use, the system 100 can be coupled to a surface of a user via the adhesive portion 114 with the frame 140 in the first frame configuration and the patch assembly 102 in the first patch configuration. The user can handle the system 100 to couple the system 100 to the patient via gripping the frame 140, which does not include adhesive on a top or bottom surface. With the patch assembly 102 coupled to the user via the adhesive portion 114, the frame 140 can be separated from the patch assembly 102 via breaking the connectors 150. For example, the frame 140 can be pulled away from the surface of the user and the patch assembly 102 with a force great enough to break the interfaces between each of the connectors 150 and the patch assembly 102. The force used to break each of the connectors 150 can be sufficiently low such that pulling the frame 140 away from the patch assembly 102 breaks the connectors 150 (e.g., at the interface between each of the connectors 150 and the patch assembly 102) but does not disrupt the adhesive interface between the adhesive portion 114 and the surface of the user such that the patch assembly 102 remains coupled to the surface of the user during and after the separation of the frame 140 from the patch assembly 102. The patch assembly 102 can then transition from the first patch configuration to the second patch configuration while remaining coupled to the surface of the patient via the adhesive portion 114.

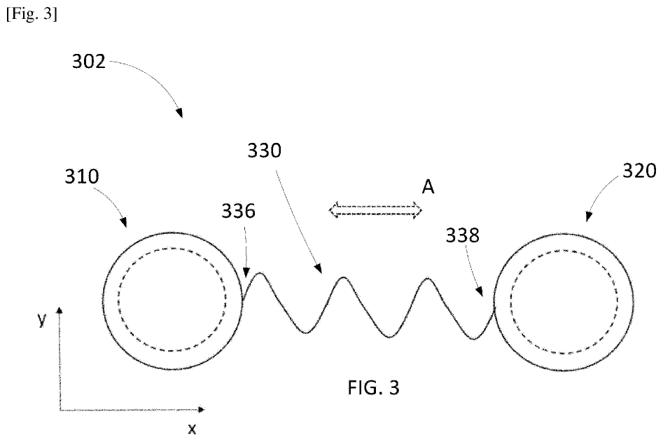

FIG. 2 is a schematic illustration of a cross-section of a system 200. The system 200 can be the same or similar in structure and/or function to any of the systems described herein, such as the system 100 described above. For example, the system 200 includes a patch assembly 202, a frame assembly 240, and a group of connectors 250. The patch assembly 202 can be disposed within an opening 242 of the frame assembly 240. The patch assembly 202, the frame assembly 240, and the group of connectors 250 can be the same or similar in structure and/or function to the patch assembly 102, the frame 140, and the group of connectors 150 described above with reference to the system 100. The system 200 also includes a protective layer 260.

The frame assembly 240 can have a first frame configuration in which the frame assembly 240 is coupled to the patch assembly 202 via the group of connectors 250 and a second frame configuration in which the group of connectors 250 are broken and the frame assembly 240 is separated from the patch assembly 202. The patch assembly 202 can have a first patch configuration and a second patch configuration. For example, the patch assembly 202 or a portion of the patch assembly 202 may have a different shape (e.g., outer profile) and/or a different length in the first patch configuration compared to the second patch configuration. When the frame assembly 240 is in the first frame configuration relative to the patch assembly 202, the frame assembly 240 can maintain the patch assembly 202 in the first patch configuration via the group of connectors 250. The frame assembly 240 can be substantially inelastic. For example, the frame assembly 240 can be inelastic along its longitudinal axis such that, when in the first frame configuration when the frame assembly 240 is coupled to the patch assembly 202, the frame assembly 240 can prevent the patch assembly 202 from changing shape via the group of connectors 250. For example, the frame assembly 240 can prevent the patch assembly 202 from transitioning between the first patch configuration and the second patch configuration.

As shown in FIG. 2, the patch assembly 202 includes a housing 206 and an adhesive portion 214. In some embodiments, the housing 206 can include an upper housing portion. In some embodiments, the housing 206 can include an upper housing portion and/or a lower housing portion. The frame assembly 240 includes a top layer 244. The top layer 244 is coupled to the patch assembly 202 in a first frame configuration via the connectors 250. For example, the top layer 244 can be coupled to the housing 206 via the connectors 250. In some embodiments, the top layer 244, the connectors 250, and the housing 206 can be formed of the same material. In some embodiments, the top layer 244, the connectors 250, and the housing 206 can be monolithically or integrally formed (e.g., formed of one sheet of material). In a second frame configuration, the frame assembly 240 can be separated from the patch assembly 202 via breaking the connectors 250.

The protective layer 260 is configured to be coupled to the patch assembly 202 via the adhesive portion 214. The protective layer 260 is shaped and sized to contact and protect the bottommost surface of the frame assembly 240 prior to use of the system 200 (e.g., during storage). The protective layer 260 can be configured to protect at least the adhesive portion 214 and/or any hydrogel portion of the underside of the patch assembly 202. The protective layer 260 can be separated from the frame assembly 240 and the patch assembly 202 via, for example, peeling the protective layer 260 away from the adhesive portion 214. After the protective layer 260 is separated from the frame assembly 240 and the patch assembly 202, the frame assembly 240 and the patch assembly 202 can be coupled to a surface (e.g., a skin) of a user via the adhesive portion 214. In some embodiments, the protective layer 260 is configured to remain coupled to a portion of the frame assembly 240 (e.g., via being bound or glued to an end of the frame assembly 240 forming a hinge portion) after the protective layer 260 is separated from the adhesive portion 214 of the patch assembly 202 such that the patch assembly 202 can be coupled to a surface of the user via the adhesive portion 214 with the protective layer 260 coupled to a portion of the frame assembly 240, as described in further detail herein.

In some embodiments, the frame assembly 240 can optionally include an adhesive layer 246 and a liner layer 248. The adhesive layer 246 can be disposed between the top layer 244 and the liner layer 248. Although not shown, in some embodiments, the adhesive layer 246 and the adhesive portion 214 can be formed as one layer that is separated or separable at an interface between the frame assembly 240 and the patch assembly 202 (e.g., via breaking the group of connectors 250). In some embodiments, each connector from the group of connectors 250 can also include an adhesive portion such that the adhesive layer 246, the adhesive portion of each of the connectors 250, and the adhesive portion 214 can be formed of a monolithic or unitary adhesive layer. The liner layer 248 can be configured to prevent the adhesive layer 246 from being exposed to the protective layer 260 and/or a surface of the user.

In some embodiments, each connector 250 can include a portion of the top layer 244 and/or the housing 206 in addition to another material. For example, in some embodiments, the frame assembly 240 can include one or more additional layers or portions of material between the top layer 244 and the liner layer 248 and/or between the top layer 244 and the adhesive layer 246. A portion of each of the connectors 250 (e.g., a weakest portion) can be formed by the same material as the additional layer or portion of material of the frame assembly 240 (e.g., monolithically or integrally formed as one piece with the additional layer or portion of the frame assembly 240). For example, the top layer 244 and/or the housing 206 can be formed of non-woven cotton and the one or more additional layers or portions of material can be formed of, for example, metal such as copper shaped to break at a particular location (e.g., via including one or more cut outs). In some embodiments, the patch assembly 202 can also include such an additional layer or portion of the same material as the portion of each of the connectors that is formed of a material different from the top layer 244 and/or the housing 206.

In some embodiments, the patch assembly 202 can optionally include an electronics subassembly 204. The electronics subassembly 204 can include any suitable electronic components such as, for example, one or more electrodes, a processor, a memory, and/or an energy storage device. In some embodiments, the housing 206 can be configured such that some or all of the electronic components of the electronics subassembly 204 are disposed between an upper housing portion and a lower housing portion of the housing 206. In some embodiments, the lower housing portion can define at least one opening through which an electrode of the electronics subassembly 204 can contact a surface of a user. In some embodiments, the adhesive portion 214 can define at least one opening through which an electrode of the electronics subassembly 204 can contact a surface of a user.

In use, with the frame in the first frame configuration and the patch in the first patch configuration, the protective layer 260 can be separated from the bottommost surface of the frame assembly 240 (e.g., from the top layer 244 or the optional liner layer 248) and the adhesive portion 214 of the patch assembly 202 (e.g., via being peeled). The frame assembly 240 and the patch assembly 202 can then be coupled to a surface of the user via the adhesive portion 214. The user can handle the system 200 to couple the system to the patient via gripping the frame assembly 240, which does not include adhesive on a top or bottom surface. With the patch assembly 202 coupled to the user via the adhesive portion 214 in the first patch configuration, the frame assembly 240 can be separated from the patch assembly 202 via breaking the group of connectors 250. For example, the frame 240 can be pulled away from the surface of the user with a force great enough to break the interfaces between each of the connectors 250 and the patch assembly 202. The force used to break each connector from the group of connectors 250 can be sufficiently low such that pulling the frame 240 away from the patch assembly 202 breaks the connectors 250 (e.g., at the interface between each of the connectors 250 and the patch assembly 202) but does not disrupt the adhesive interface between the adhesive portion 214 and the surface of the user such that the patch assembly 202 remains coupled to the surface of the user during and after the separation of the frame 240 from the patch assembly 202. The patch assembly 202 can then transition to the second patch configuration.

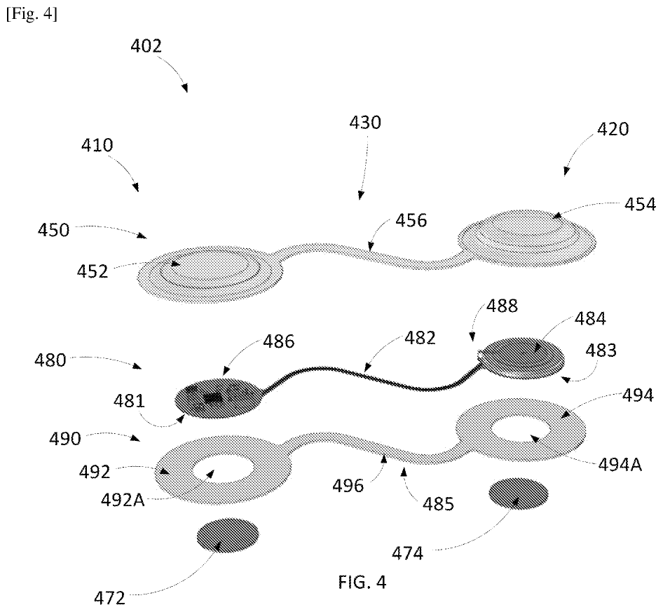

In some embodiments, any of the patch assemblies described herein can be the same or similar to any of the patch assemblies described in International Patent Application No. PCT/JP2020/002521, filed Jan. 24, 2020, titled “Elastic Wearable Sensor” (hereinafter “the '521 application”) and/or U.S. Provisional Patent Application No. 62/796,435, filed Jan. 24, 2019, titled “Elastic Wearable Sensor,” each of which is incorporated by reference herein in its entirety. For example, any of the patch assemblies described herein can include patch assembly 302 shown in FIG. 3, which corresponds to FIG. 2A of the '521 application. FIG. 3 is a schematic illustration of a top view of the patch assembly 302. The patch assembly 302 can include a first assembly 310, a second assembly 320, and a connecting member 330. As shown in FIG. 3, the connecting member 330 includes a first end 336 and a second end 338. The connecting member 330 is coupled to the first assembly 310 via the first end 336 and to the second assembly 320 via the second end 338. The connecting member 330 is configured to transition between a first configuration (shown in FIG. 3) and a second configuration in which the first assembly 310 and the second assembly 320 are a different distance away from each other than in the first configuration. For example, when the patch assembly 302 is coupled to a patient's skin, a force (e.g., due to deformation due to skin flexing or tension) may be applied to the first assembly 310 and/or the second assembly 320 in either direction represented by the double-ended arrow A (e.g., in the X-direction) such that the length of the connecting member 330 from the first end 336 to the second end 338 is increased or decreased and the connecting member 330 is compressed or expanded. In some implementations, a force may be applied to the first assembly 310 and/or the second assembly 320 in any direction in the X-Y plane such that the length of the connecting member 330 from the first end 336 to the second end 338 is increased or decreased and the connecting member 330 is compressed or expanded. In some embodiments, the connecting member 330 can be biased toward the first configuration of the connecting member 330.

A frame assembly, such as any of the frame assemblies described herein, can be coupled to the patch assembly 302 via connectors coupled to the first assembly 310, the second assembly 320, and/or the connecting member 330 such that the frame assembly can maintain the patch assembly 302 in a first patch configuration. For example, the frame assembly can maintain an intended distance between the first assembly 310 and the second assembly 320 such that, when the patch assembly 302 is coupled to a user's skin via adhesive, the first assembly 310 and the second assembly 320 are separated by the intended distance. For instance, the first assembly 310 and the second assembly 320 may each include an electrode, and the frame assembly may be configured to maintain an electrode to electrode distance of the patch assembly 302 such that the electrodes can be properly spaced apart when the patch assembly 302 is coupled to a user's skin. Additionally, in some embodiments, an opening defined by the frame assembly can be shaped to correspond to an outer profile of at least a portion of the patch assembly disposed within the opening. For example, the connecting member 330 may have a sinusoidal shape and the opening defined by the frame assembly can have a sinusoidal shape corresponding to an outer profile of at least a portion of the connecting member 330.

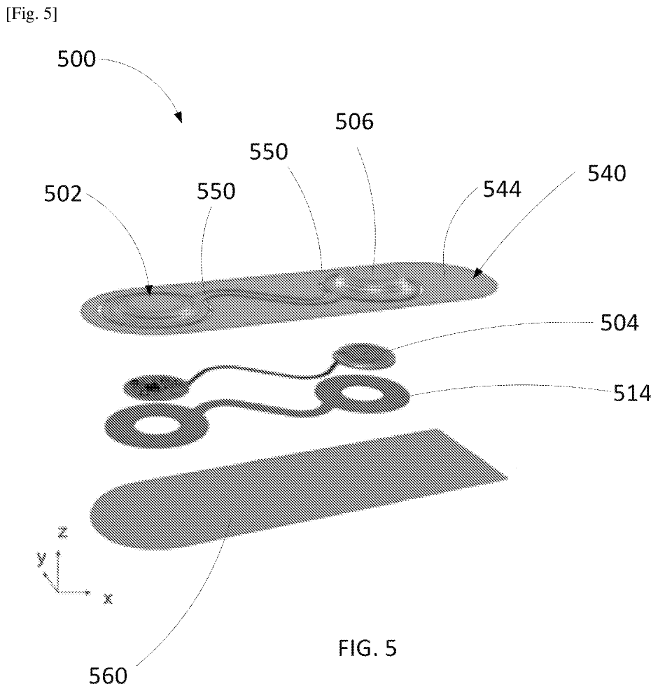

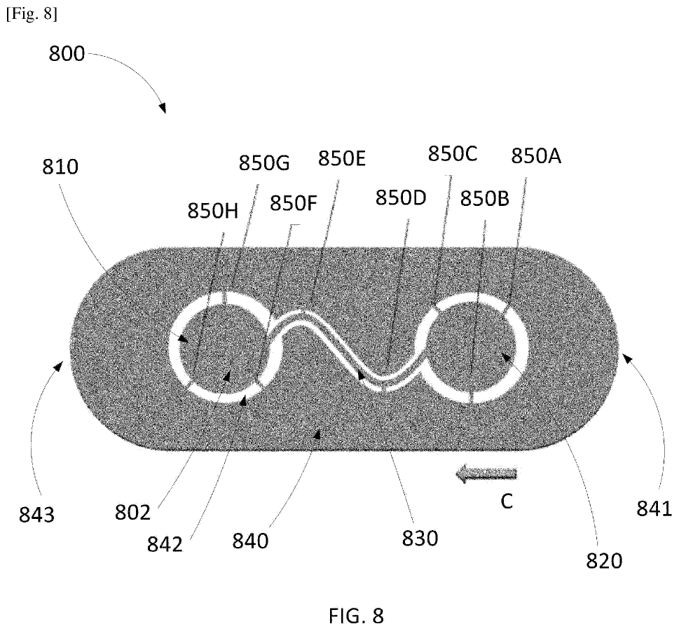

In some embodiments, any of the patch assemblies described herein can include patch assembly 402 shown in FIG. 4, which corresponds to FIG. 7 of the '521 application. FIG. 4 is a perspective exploded view of a patch assembly 402. The patch assembly 402 can be the same or similar in structure and/or function to any of the systems described herein, such as, for example, the patch assembly 102, the patch assembly 202, or the patch assembly 302. The patch assembly 402 includes a first assembly 410, a second assembly 420, and a connecting member 430 that can be the same or similar in structure and/or function, for example, to the first assembly 310, the second assembly 320, and/or the connector 330, respectively. The first assembly 410 includes a first upper housing 452, a portion 486 of a composite assembly 480, a first lower housing 492, and a first adhesive portion (not shown). The composite assembly 480 can be included in and/or otherwise form an integrated circuit (IC), a printed circuit board (PCB) assembly including a printed circuit board, an application-specific integrated circuit (ASIC), or any other suitable electrical circuit structure. For example, the portion 486 can include any suitable electronic components (e.g., a processor and a memory). The first lower housing 492 defines an opening 492A such that an electrode 481 disposed on a bottom side of the portion 486 is accessible through the opening 492A. The first adhesive portion can also define an opening (e.g., similar in size and shape to the opening 492A) such that the electrode 481 disposed on a bottom side of the portion 486 is accessible through the opening 492A. The first assembly 410 also includes a hydrogel portion 472.

The second assembly 420 includes a second upper housing 454, a portion 484 of the composite assembly 480, a second lower housing 494, and a second adhesive portion (not shown). The portion 484 can include any suitable electronic components (e.g., an energy storage device such as a coin cell battery). The second lower housing 494 defines an opening 494A such that an electrode 483 disposed on a bottom side of the portion 484 is accessible through the opening 494A. The second adhesive portion can also define an opening (e.g., similar in size and shape to the opening 494A) such that the electrode 483 disposed on a bottom side of the portion 484 is accessible through the opening 492A. The second assembly 420 also includes a hydrogel portion 474.

In some implementations, the composite assembly 480 includes a tab contact 488. The tab contact 488 can be integrally formed with the composite board of the composite assembly 480 and can be folded to contact the top of the energy storage device of the portion 484 as shown in FIG. 4. In some implementations, the energy storage device can be coupled to the composite board of the composite assembly 480 via a conductive adhesive. In some implementations, contacts of the energy storage device can be coupled to the composite board via spot welding.

The connecting member 430 includes a third upper housing 456, a portion 482 of the composite assembly 480, a third lower housing 496, and a third adhesive portion (not shown). The third lower housing 496 has a skin-facing surface 485 along the full length of the portion 482. The portion 482 can include a composite board including an insulator and at least one conductive trace (e.g., a flexible printed circuit board). The insulator can include, for example, polyimide. The at least one conductive trace can include, for example, copper. In some implementations, the composite board can include a polyimide with double-sided copper conductors. In some implementations, the portion 482 can include multiple layers (e.g., two, three, or more layers), each layer including at least one conductive trace. In some implementations, the portion 482 can include multiple layers including at least one conductive trace, each layer including at least one conductive trace coupled to another layer including at least one conductive trace via an insulative layer. In some implementations, the third adhesive portion can cover the entire skin-facing surface 485 of the third lower housing 496. In some implementations, the patch assembly 402 includes three conductive traces extending from the first assembly 410 to the second assembly 420. For example, a first conductive trace can extend from a positive side of the energy storage device of the portion 484 to the portion 486, a second conductive trace can extend from a negative side of the energy storage device of the portion 484 to the portion 486, and the third conductive trace can extend from the electrode 483 to the portion 486. Similarly as described above with reference to the connecting member 130, in some implementations the connecting member 430 (and/or the portion 482) may have a thickness equal to or less than 100 μm. In some implementations, the height of the connecting member 430 (and/or the portion 482) can be, for example, equal to or less than 36 μm. In some implementations, the spring constant of the connecting member 430 (and/or the portion 482) (in the X-direction) can increase proportionally to a cube of the thickness of the connecting member 430 (and/or the portion 482) and linearly with respect to the height of the connecting member 430 (and/or the portion 482).

As shown in FIG. 4, the first upper housing 452, the second upper housing 454, and the third upper housing 456 can collectively form a cover layer 450. The first lower housing 492, the second lower housing 494, and the third lower housing 496 can collectively form a bottom layer 490. The bottom layer 490 can be coupleable to a surface of a skin via the first adhesive portion, the second adhesive portion, and/or the third adhesive portion such that the bottom layer 490 secures the composite assembly 480 to the surface of the skin. In some implementations, the cover layer 450 (including the first upper housing 452, the second upper housing 454, and the third upper housing 456) can be monolithically or integrally formed. In some implementations, the bottom layer 490 (including the first lower housing 492, the second lower housing 494, and the third lower housing 496) can be monolithically or integrally formed. In some embodiments, the first adhesive portion, the second adhesive portion, and the third adhesive portion can be included in a continuous adhesive layer. The continuous adhesive layer can be shaped and sized similarly to the bottom layer 490 and disposed on the bottom surface of the bottom layer 490.

A frame assembly, such as any of the frame assemblies described herein, can be coupled to the patch assembly 402 via connectors coupled to the first assembly 410, the second assembly 420, and/or the connecting member 430 such that the frame assembly can maintain the patch assembly 402 in a first patch configuration. For example, the frame assembly can maintain an intended distance between the first assembly 410 and the second assembly 420 and a particular shape of the connecting member 430 such that, when the patch assembly 402 is coupled to a user's skin via adhesive, the first assembly 410 and the second assembly 420 are separated by the intended distance and the connecting member 430 conformally couples to the skin of the user. For instance, the first assembly 410 and the second assembly 420 may each include an electrode, and the frame assembly may be configured to maintain an electrode to electrode distance of the patch assembly 402 such that the electrodes can be properly spaced apart when the patch assembly 402 is coupled to a user's skin. Additionally, in some embodiments, an opening defined by the frame assembly can be shaped to correspond to an outer profile of at least a portion of the patch assembly disposed within the opening. For example, the connecting member 430 may have a sinusoidal shape and the opening defined by the frame assembly can have a sinusoidal shape corresponding to an outer profile of at least a portion of the connecting member 430.

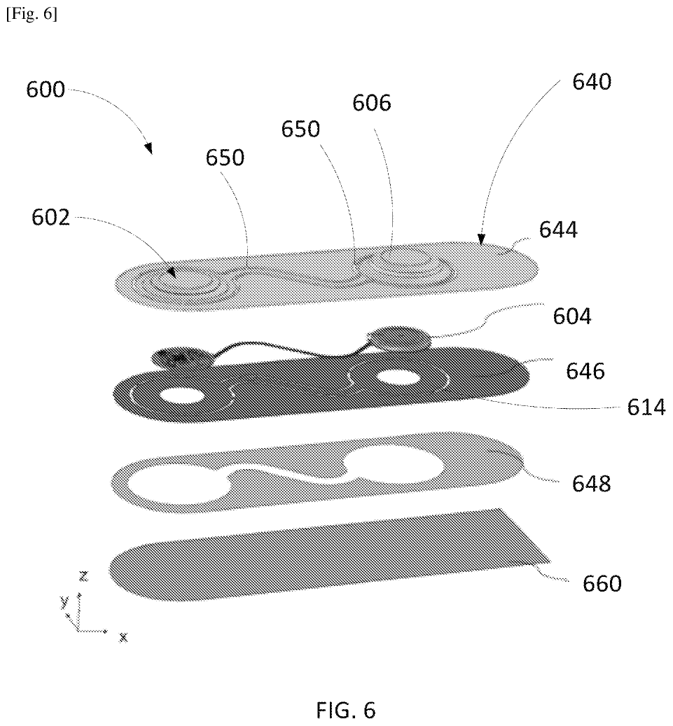

FIG. 5 is an exploded view of a system 500. The system 500 can be the same or similar in structure and/or function to any of the systems described herein. For example, the frame assembly 540 can be the same or similar in structure and/or function to any of the frames or frame assemblies described herein. For example, the frame assembly 540 includes a top layer 544. The patch assembly 502 can be the same or similar in structure and/or function to any of the patch assemblies described herein. For example, the patch assembly 502 includes a housing 506, an electronics assembly 504, and an adhesive portion 514. In some embodiments, the adhesive portion 514 can form or include a lower housing portion of the patch assembly 502. In some embodiments, the adhesive portion 514 can be cut to the appropriate shape and size for coupling the patch assembly 502 to a user's skin. In some embodiments, the adhesive portion 514 can be formed via patterned deposition. As shown in FIG. 5, the adhesive portion 514 can define openings such that electrodes of the patch assembly 502 are accessible via the openings and can contact a surface of a user when the patch assembly 502 is coupled to the user via the adhesive portion 514. In some embodiments, the electronics assembly 504 may be the same or similar in structure and/or function to the composite assembly 480 shown and described with respect to FIG. 4. The frame assembly 540 is configured to be coupled to the patch assembly 502 via a set of connectors 550. The system 500 also includes a protective layer 560.

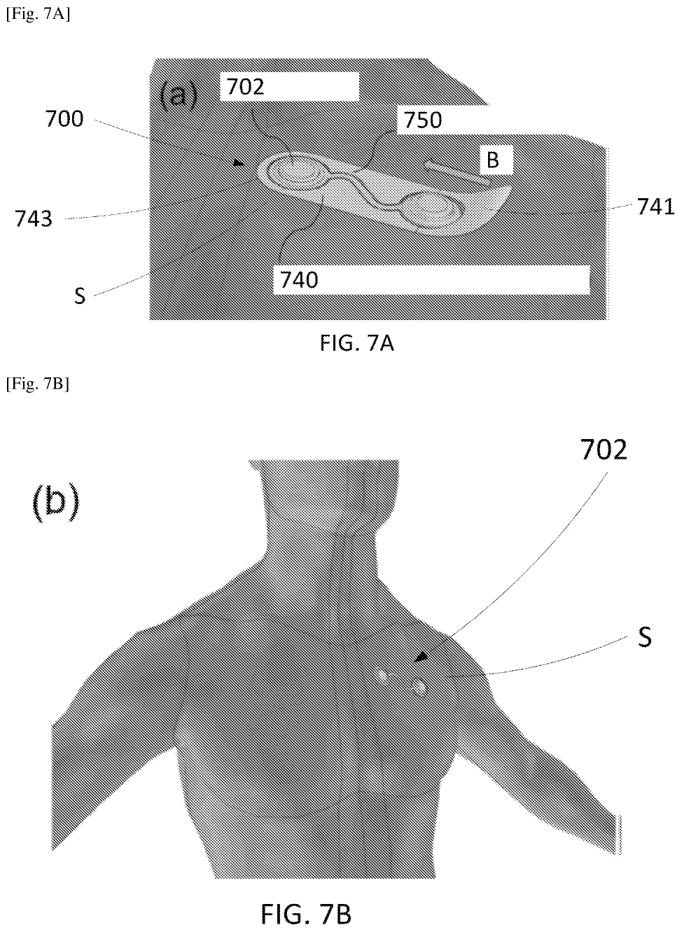

FIG. 6 is an exploded view of a system 600. The system 600 can be the same or similar in structure and/or function to any of the systems described herein. For example, the frame assembly 640 can be the same or similar in structure and/or function to any of the frames or frame assemblies described herein. For example, the frame assembly 640 includes a top layer 644, an adhesive layer 646, and a liner layer 648. The patch assembly 602 can be the same or similar in structure and/or function to any of the patch assemblies described herein. For example, the patch assembly 602 includes a housing 606, an electronics assembly 604, and an adhesive portion 614. In some embodiments, the adhesive portion 614 can form or include a lower housing portion of the patch assembly 602. As shown in FIG. 6, the adhesive portion 614 can define openings such that electrodes of the patch assembly 602 are accessible via the openings and can contact a surface of a user when the patch assembly 602 is coupled to the user via the adhesive portion 614. In some embodiments, the electronics assembly 604 may be the same or similar in structure and/or function to the composite assembly 480 shown and described with respect to FIG. 4. The frame assembly 640 is configured to be coupled to the patch assembly 602 via connectors 650. Each of the connectors 650 can include a housing portion and an adhesive portion such that the top layer 644, the housing 606, and the housing portion of each connector 650 can be formed as a single unitary layer, and the adhesive layer 646, the adhesive portion of the connectors 650, and the adhesive portion 614 can be formed as a single unitary layer. In some embodiments, the adhesive portion of each connector 650 can be omitted such that the connectors 650 break more easily. The system 600 also includes a protective layer 660.

FIG. 7A is an illustration of a system 700 disposed on a surface S (e.g., skin) of a user. The system 700 can be the same or similar to any of the systems described herein. For example, the system 700 can include a frame assembly 740 and a patch assembly 702. The patch assembly 702 is coupled to the frame assembly 740 via connectors 750. The frame assembly 740 has a first end 741 and a second end 743. As shown in FIG. 7A, the system 700 can be disposed on the surface S of the user such that the patch assembly 702 is in a first patch configuration and is coupled to the surface S via an adhesive portion (not shown). The frame assembly 740 can then be separated from the patch assembly 702 via pulling the first end 741 of the frame assembly 740 in the direction of arrow B (e.g., toward the second end 743 of the frame assembly 740) such that each of the connectors 750 breaks and the frame assembly 740 is separated from the patch assembly 702. As shown in FIG. 7B, which is an illustration of the patch assembly 702 disposed on the surface S of the user, the patch assembly 702 can remain disposed on the surface S after the frame assembly 740 is separated from the patch assembly 702. The patch assembly 702, having been separated from the frame assembly 740, can then transition between the first patch configuration and a second patch configuration to accommodate skin deformations of the user and reduce stress at the skin-adhesive interface.

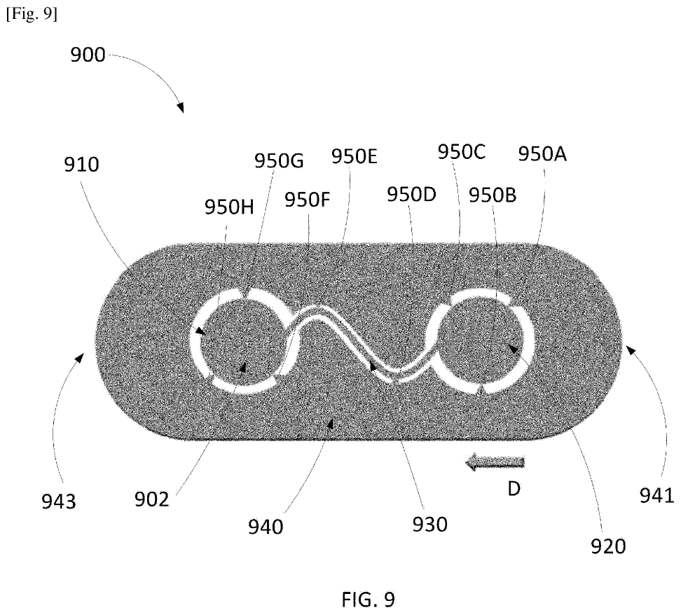

FIG. 8 is a top view of a system 800 including a patch assembly 802 and a frame assembly 840. The system 800 can be the same or similar in structure and/or function to any of the systems described herein. The patch assembly 802 is in a first patch configuration and is coupled to the frame assembly 840 via eight connectors 850A-850H. Specifically, the patch assembly 802 is coupled to the frame assembly 840 via a first connector 850A, a second connector 850B, a third connector 850C, a fourth connector 850D, a fifth connector 850E, a sixth connector 850F, a seventh connector 850G, and an eighth connector 850H. Although the system 800 is shown as including eight connectors, in some embodiments the system can include any suitable number of connectors.

The patch assembly 802 can be the same or similar in structure and/or function to any of the systems described herein. For example, the patch assembly 802 includes a first assembly 810, a second assembly 820, and a connecting member 830. As shown in FIG. 8, connectors 850A-850H can couple each of the first assembly 810, the second assembly 820, and the connecting member 830 to the frame assembly 840. Specifically, the first connector 850A, the second connector 850B, and the third connector 850C can coupled the second assembly 820 to the frame assembly 840. The fourth connector 850D and the fifth connector 850E can couple the connecting member 830 to the frame assembly 840. The sixth connector 850F, the seventh connector 850G, and the eighth connector 850H can couple the first assembly 810 to the frame assembly 840.

As shown in FIG. 8, the connecting member 830 has a first sinusoidal shape having a first frequency in the first configuration of the patch assembly 802. The connecting member 830 can also have a second sinusoidal shape having a second frequency in a second configuration of the patch assembly 802 (not shown in FIG. 8). The second frequency can be different from the first frequency. For example, the connecting member 830 may be shorter or longer in overall length in the second frequency compared to the first configuration. The opening 842 defined by the frame assembly 840 can include an opening portion having a sinusoidal shape having the first frequency such that the connecting member 830 of the patch assembly 802 can be disposed within the opening 842 when the patch assembly 802 is in the first configuration.

The frame assembly 840 has a first end 841 and a second end 843. In use, the system 800 can be disposed on a surface (e.g., skin) of a user such that the patch assembly 802 is in the first patch configuration and coupled to the surface via an adhesive portion (not shown). A user can grip the frame assembly 840 to apply the system 800 to the surface of the user without touching an adhesive portion of the patch assembly 802. The frame assembly 840 can then be separated from the patch assembly 802 via pulling the first end 841 of the frame assembly 840 in the direction of arrow C (e.g., toward the second end 843 of the frame assembly 840) such that each of the connectors 850A-850H breaks (e.g., due to a shear force) and the frame assembly 840 is separated from the patch assembly 802. A portion of the frame assembly 840 (e.g., an end such as the first end 841) can function as a handle during the separation of the frame assembly 840 from the patch assembly 802. The patch assembly 802 can remain disposed on the surface after the frame assembly 840 is separated from the patch assembly 802. The patch assembly 802, having been separated from the frame assembly 840, can then transition between a first patch configuration and a second patch configuration to accommodate skin deformations of the user and reduce stress at the skin-adhesive interface.

As shown in FIG. 8, each connector 802 is arranged such that each laterally-extending plane disposed perpendicular to a longitudinal axis of the patch assembly includes either a single connector or no connector. As a result, when the first end 841 of the frame assembly 840 is pulled relative to the patch assembly 802 in the direction of arrow C (e.g., the tear-off direction), each connector 840 can break sequentially (i.e., only one connector 850A-850H is broken at a time) such that the connectors 840 are broken in series. Since the force applied in the tear-off direction can be applied to one connector 850A-850H at a time, the force applied to the patch assembly 802 is reduced compared to the force applied to the patch assembly 802 if two or more connectors 850A-850H are pulled and/or broken simultaneously. Reducing the force applied to the patch assembly 802 reduces stress on and disruption of the skin-adhesive interface between the adhesive portion of the patch assembly and the skin of the user.

As shown in FIG. 8, each of the connectors 850A-850H extends non-parallel to a longitudinal axis of the patch assembly 802. For example, some of the connectors 850 extend perpendicularly and some connectors 850A-850H extend at an angle that is neither parallel nor perpendicular to a longitudinal axis of the patch assembly 802. Thus, when a separating force is applied to the connectors 850A-850H when the frame assembly 840 is pulled relative to the patch assembly 802 in the direction of arrow C, stress can be reduced at the skin-adhesive interface between an adhesive portion (not shown) of the patch assembly 802 and a skin of a user.

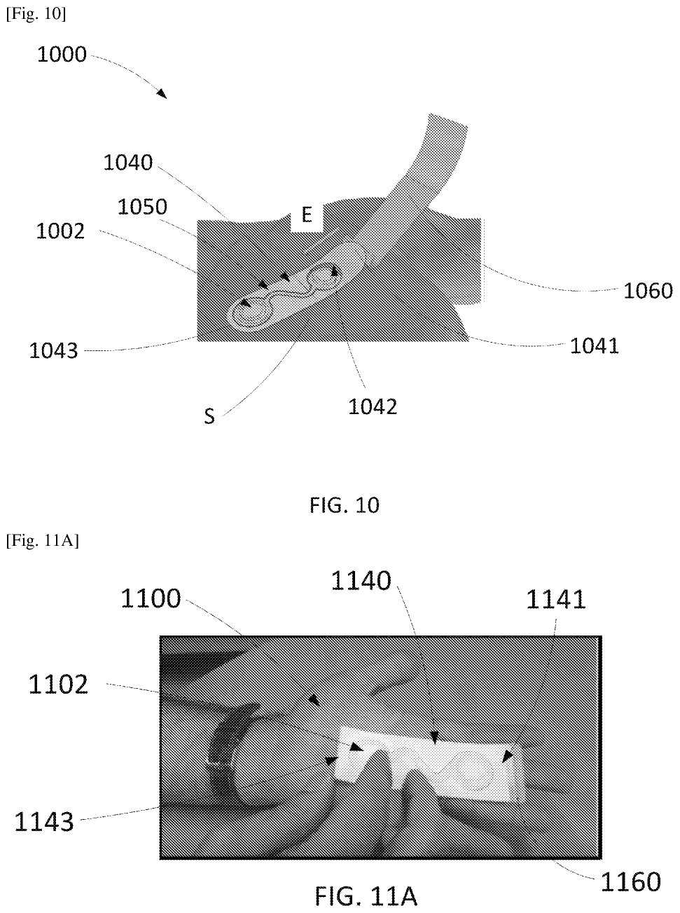

Although the connectors 850A-850H of the system 800 are shown as being rectangular, the systems described herein can include connectors having any suitable shape. For example, FIG. 9 is a top view of a system 900 including a patch assembly 902 and a frame assembly 940. The system 900 can be the same or similar in structure and/or function to any of the systems described herein. The patch assembly 902 is in a first configuration of the patch assembly 902 and is coupled to the frame assembly 940 via eight connectors 950A-950H. Specifically, the patch assembly 902 is coupled to the frame assembly 940 via a first connector 950A, a second connector 950B, a third connector 950C, a fourth connector 950D, a fifth connector 950E, a sixth connector 950F, a seventh connector 950G, and an eighth connector 950H.

Each of the connectors 950A-950H can have a triangular shape such that the connectors 950A-950H taper from the frame assembly 940 to the patch assembly 902. Thus, since the portion of each connector 950A-950H adjacent the patch assembly 902 is the most narrow portion of each connector 950A-950H, the connectors 950A-950H are likely and/or configured to separate from the patch assembly 902 when a first end 941 of the frame assembly 940 is pulled in the direction of arrow D (e.g., toward a second end 943 of the frame assembly 940).

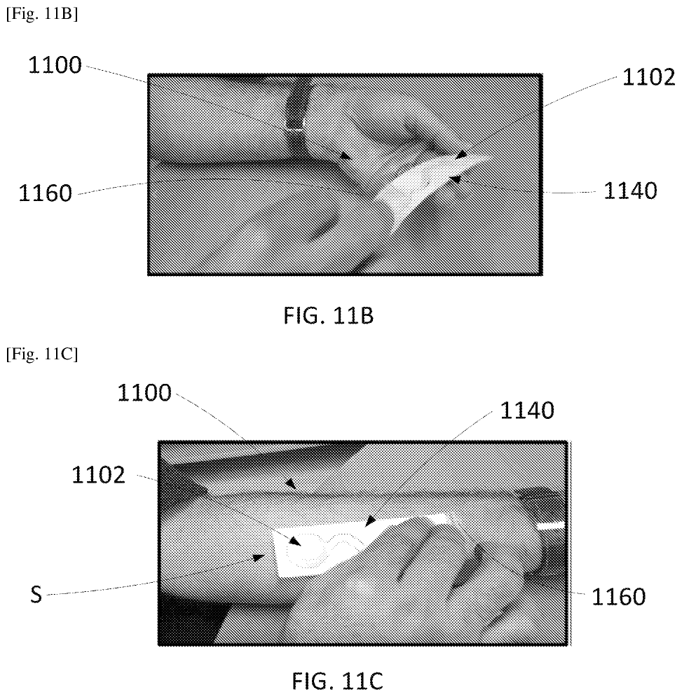

In some embodiments, a protective layer can be configured to remain attached to a frame assembly after being separated from a patch assembly. For example, FIG. 10 is an illustration of a system 1000 coupled to a surface S (e.g., skin) of a patient. The system 1000 can be the same or similar in structure and/or function to any of the systems described herein. For example, the system 1000 can include a frame assembly 1040, a patch assembly 1002, and a protective layer 1060. The frame assembly 1040 can define an opening 1042 within which the patch assembly 1002 can be disposed. The frame assembly 1040 and the patch assembly 1050 can be coupled to each other via connectors 1050. The protective layer 1060 can be coupled and/or bonded to a portion of the frame assembly 1040 via any suitable type of bonding such that, when a force (e.g., a pulling or peeling force) is applied to the protective layer 1060 to separate the protective layer 1060 from the patch assembly 1002 and the bottommost layer of the frame assembly 1040, the protective layer 1060 is separated from the patch assembly 1002 and a portion of the bottommost layer of the frame assembly 1040, but remains coupled to a portion of the frame assembly 1040 (e.g., the first end 1041 of the frame assembly 1040). For example, the protective layer 1060 can be coupled to the patch assembly 1002 via a removable adhesive and to a portion of the frame assembly 1040 (e.g., the first end 1041 of the frame assembly 1040) via a non-removable adhesive.

As shown in FIG. 10, prior to disposing the frame assembly 1040 and the patch assembly 1002 on the surface S, the protective layer 1060 can be pulled away from the frame assembly 1040 and the patch assembly 1002 such that the protective layer 1060 does not contact the patch assembly 1002 but remains coupled to the first end 1041 of the frame assembly 1040. The patch assembly 1002 and the frame assembly 1040 can then be coupled to the surface S and secured to the surface S via an adhesive portion of the patch assembly 1002. The protective layer 1060 and/or the first end 1041 can then be pulled in the direction of arrow E (e.g., toward the second end 1043 of the frame assembly 1040) such that the connectors 1050 are broken and the frame assembly 1040 is separated from the patch assembly 1002, leaving the patch assembly 1002 on the surface S.

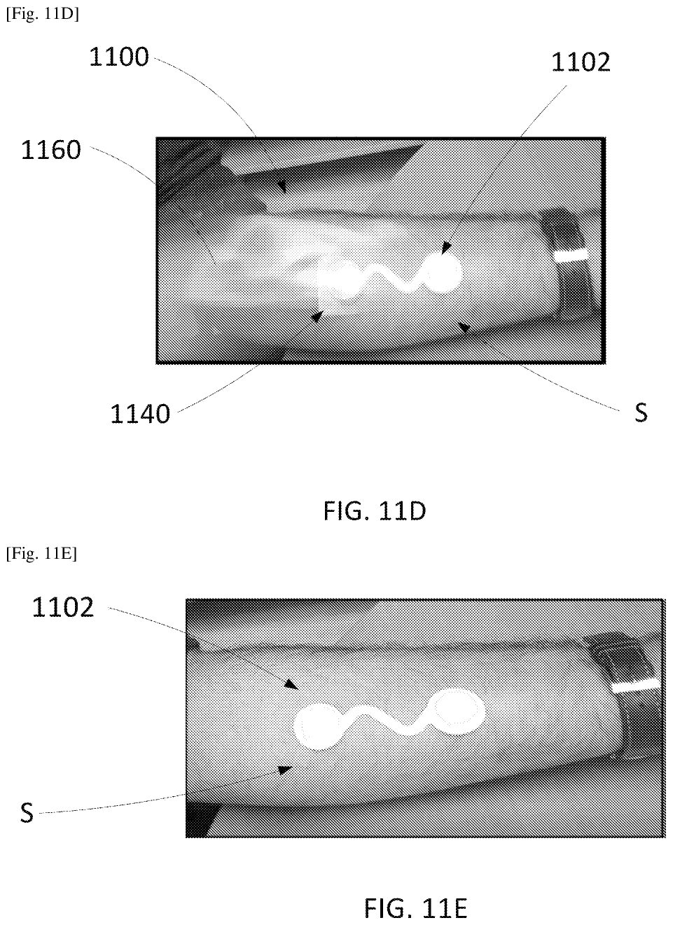

FIGS. 11A-11E are photographs illustrating the steps of using a system 1100, which can be the same or similar in structure and/or function to any of the systems described herein. As shown in FIG. 11A, the system 1100 includes a frame assembly 1140, a patch assembly 1102, and a protective layer 1160. The frame assembly 1140 can define an opening within which the patch assembly 1102 can be disposed. The frame assembly 1140 and the patch assembly 1150 can be coupled to each other via connectors (such as any of the connectors described herein). The protective layer 1160 can be coupled and/or bonded to a portion of the frame assembly 1140 via any suitable type of bonding such that, when a force (e.g., a pulling or peeling force) is applied to the protective layer 1160 to separate the protective layer 1160 from the patch assembly 1102 and the bottommost layer of the frame assembly 1140, the protective layer 1160 is separated from the patch assembly 1102 and a portion of the bottommost layer of the frame assembly 1140, but remains coupled to a portion of the frame assembly 1140 (e.g., the first end 1141 of the frame assembly 1140). For example, the protective layer 1160 can be coupled to the patch assembly 1102 via a removable adhesive and to a portion of the frame assembly 1140 (e.g., the first end 1141 of the frame assembly 1140) via a non-removable adhesive.

As shown in FIG. 11B, prior to disposing the frame assembly 1140 and the patch assembly 1102 on the surface S, the protective layer 1160 can be pulled away from the frame assembly 1140 and the patch assembly 1102 such that the protective layer 1160 does not contact the patch assembly 1102 but remains coupled to the first end 1141 of the frame assembly 1140. The user can grip the frame assembly 1140, avoiding any adhesive or hydrogel surfaces of the patch assembly 1102.

As shown in FIG. 11C, the patch assembly 1102 and the frame assembly 1140 can be coupled to the surface S with the patch assembly 1102 in a first patch configuration and the frame assembly 1140 coupled to the patch assembly 1102 via the connectors. The patch assembly 1102 can be secured to the surface S via an adhesive portion of the patch assembly 1102. The protective layer 1160 can remain attached to the frame assembly 1140 and serve as a reminder to the user that the frame assembly 1140 should be separated from the patch assembly 1102.

As shown in FIG. 11D, the protective layer 1160 and/or an end of the frame assembly 1140 can then be pulled away from the patch assembly 1102 such that the connectors are broken and the frame assembly 1140 is separated from the patch assembly 1102, leaving the patch assembly 1102 on the surface S.

As shown in FIG. 11E, the patch assembly 1102 can remain on the surface S. Additionally, the patch assembly 1102 can be configured to transition between the first patch configuration (i.e., the configuration of the patch assembly 1102 when coupled to the frame assembly 1140) and a second patch configuration (e.g., as a result of deformation of the surface S).

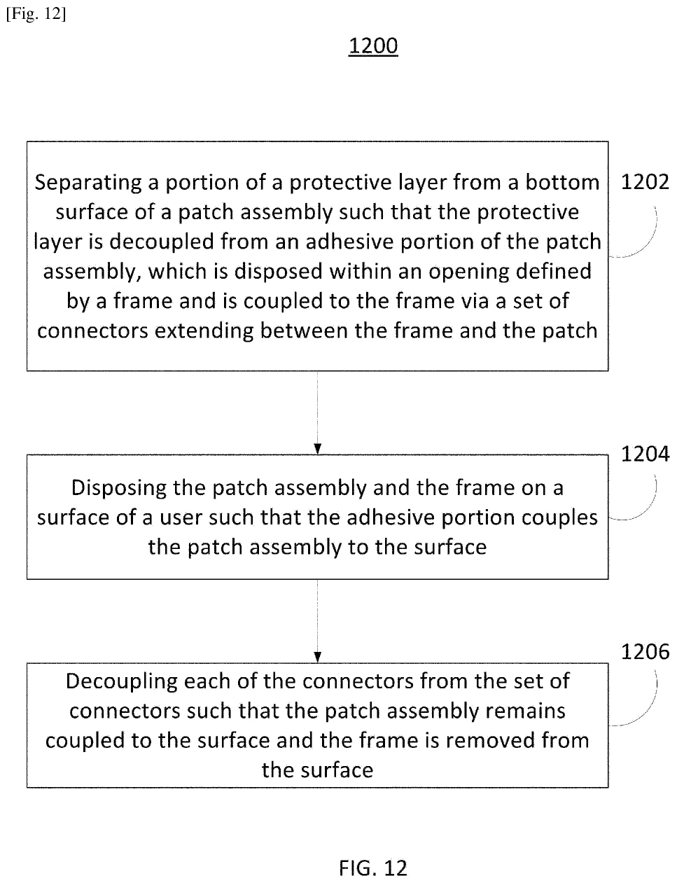

FIG. 12 is a flow chart representing a method 1200 of using any of the systems described herein. The method 1200 includes, at 1202, separating a portion of a protective layer from a bottom surface of a patch assembly such that the protective layer is decoupled from an adhesive portion of the patch assembly. The patch assembly can be disposed within an opening defined by a frame. The patch assembly is coupled to the frame via a set of connectors extending between the frame and the patch. In some embodiments, the portion of the protective layer from the bottom surface of the patch assembly can be separated such that the protective layer remains coupled to the frame (e.g., at a hinge portion).

At 1204, the patch assembly and the frame can be disposed on a surface of a user such that the adhesive portion couples the patch assembly to the surface. At 1206, each of the connectors from the set of connectors can be decoupled such that the patch assembly remains coupled to the surface and the frame is removed from the surface. In some embodiments, each of the connectors of the plurality of connectors can be decoupled by pulling a first end of the frame toward a second end of the frame such that each connector from the set of connectors breaks in series.

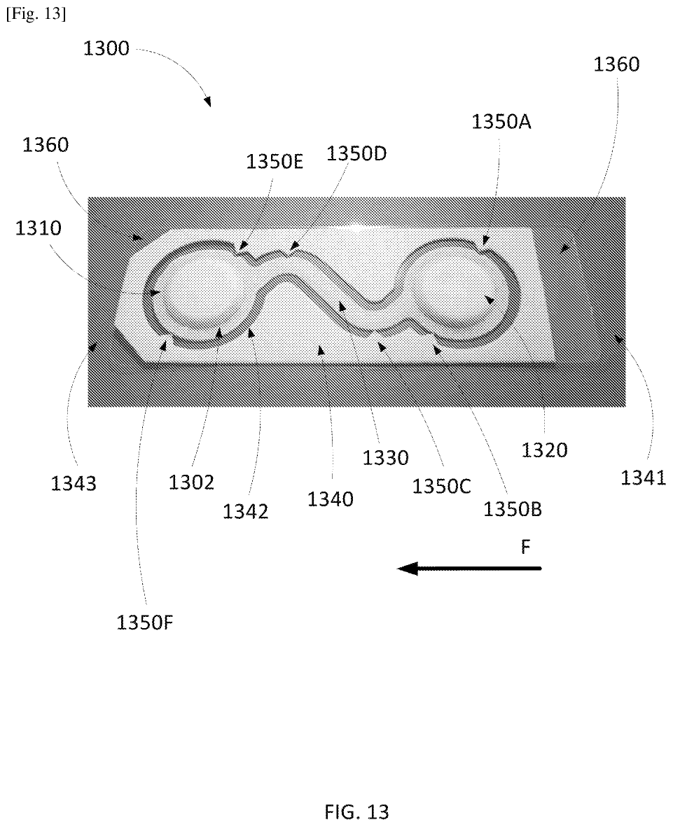

In some embodiments, a portion of each connector of a system can include a gap between a top layer of the connector and either a patch assembly or a frame assembly of the system to reduce an amount of shear force needed to separate the patch assembly from the frame assembly of the system. FIG. 13 is a top view of a system 1300 including a patch assembly 1302, a frame assembly 1340, and a protective layer 1360. The system 1300 can be the same or similar in structure and/or function to any of the systems described herein. The patch assembly 1302 is in a first patch configuration and is coupled to the frame assembly 1340 via six connectors 1350A-1350F. Specifically, the patch assembly 1302 is coupled to the frame assembly 1340 via a first connector 1350A, a second connector 1350B, a third connector 1350C, a fourth connector 1350D, a fifth connector 1350E, and a sixth connector 1350F. Although the system 1300 is shown as including six connectors, in some embodiments the system can include any suitable number of connectors.

The patch assembly 1302 can be the same or similar in structure and/or function to any of the systems described herein. For example, the patch assembly 1302 includes a first assembly 1310, a second assembly 1320, and a connecting member 1330. As shown in FIG. 13, connectors 1350A-1350F can couple each of the first assembly 1310, the second assembly 1320, and the connecting member 1330 to the frame assembly 1340. Specifically, the first connector 1350A and the second connector 1350B can coupled the second assembly 1320 to the frame assembly 1340. The third connector 1350C and the fourth connector 1350D can couple the connecting member 1330 to the frame assembly 1340. The fifth connector 1350E and the sixth connector 1350F can couple the first assembly 1310 to the frame assembly 1340.

As shown in FIG. 13, the connecting member 1330 has a first sinusoidal shape having a first frequency in the first configuration of the patch assembly 1302. The connecting member 1330 can also have a second sinusoidal shape having a second frequency in a second configuration of the patch assembly 1302 (not shown in FIG. 13). The second frequency can be different from the first frequency. For example, the connecting member 1330 may be shorter or longer in overall length in the second frequency compared to the first configuration. The opening 1342 defined by the frame assembly 1340 can include an opening portion having a sinusoidal shape having the first frequency such that the connecting member 1330 of the patch assembly 1302 can be disposed within the opening 1342 when the patch assembly 1302 is in the first configuration.

The frame assembly 1340 has a first end 1341 and a second end 1343. In use, the system 1300 can be disposed on a surface (e.g., skin) of a user such that the patch assembly 1302 is in the first patch configuration and coupled to the surface via an adhesive portion (not shown). A user can grip the frame assembly 1340 to apply the system 1300 to the surface of the user without touching an adhesive portion of the patch assembly 1302. The frame assembly 1340 can then be separated from the patch assembly 1302 via pulling the first end 1341 of the frame assembly 1340 in the direction of arrow F (e.g., toward the second end 1343 of the frame assembly 1340) such that each of the connectors 1350A-1350F breaks (e.g., due to a shear force) and the frame assembly 1340 is separated from the patch assembly 1302. A portion of the frame assembly 1340 (e.g., an end such as the first end 1341) can function as a handle during the separation of the frame assembly 1340 from the patch assembly 1302. The patch assembly 1302 can remain disposed on the surface after the frame assembly 1340 is separated from the patch assembly 1302. The patch assembly 1302, having been separated from the frame assembly 1340, can then transition between a first patch configuration and a second patch configuration to accommodate skin deformations of the user and reduce stress at the skin-adhesive interface.

As shown in FIG. 13, each connector 1302 optionally can be arranged such that each laterally-extending plane disposed perpendicular to a longitudinal axis of the patch assembly includes either a single connector or no connector. As a result, when the first end 1341 of the frame assembly 1340 is pulled relative to the patch assembly 1302 in the direction of arrow F (e.g., the tear-off direction), each connector 1340 can break sequentially (i.e., only one connector 1350A-1350F is broken at a time) such that the connectors 1340 are broken in series. Since the force applied in the tear-off direction can be applied to one connector 1350A-1350F at a time, the force applied to the patch assembly 1302 is reduced compared to the force applied to the patch assembly 1302 if two or more connectors 1350A-1350F are pulled and/or broken simultaneously. Reducing the force applied to the patch assembly 1302 reduces stress on and disruption of the skin-adhesive interface between the adhesive portion of the patch assembly and the skin of the user.

As shown in FIG. 13, each of the connectors 1350A-1350F optionally can be arranged to extend non-parallel to a longitudinal axis of the patch assembly 1302. For example, some of the connectors 1350 extend perpendicularly and some connectors 1350A-1350F extend at an angle that is neither parallel nor perpendicular to a longitudinal axis of the patch assembly 1302. Thus, when a separating force is applied to the connectors 1350A-1350F when the frame assembly 1340 is pulled relative to the patch assembly 1302 in the direction of arrow F, stress can be reduced at the skin-adhesive interface between an adhesive portion (not shown) of the patch assembly 1302 and a skin of a user.

Additionally, as shown in FIG. 13, each of the connectors 1350A-1350F includes a first portion (e.g., a top layer) including a first material and a second portion (e.g., a bottom layer) including a second material. The first portion extends only a portion of a distance between the frame assembly 1340 and the patch assembly 1302 and tapers toward the patch assembly 1302. A gap can be defined between the first portion and the patch assembly 1302. The second portion extends at least beyond the end of the first portion and connects to the patch assembly 1302. For example, the second portion can extend from the frame assembly 1340 to the patch assembly 1302 and can be shaped such that the weakest portion of each connector 1350A-1350F is a portion of the second portion that extends beyond the first portion to the patch assembly 1302. In some embodiments, the first portion can include a non-woven material (e.g., non-woven cotton) and the second portion can include a metal (e.g., copper) that is configured to break near or adjacent to the patch assembly 1302. Thus, each of the connectors 1350A-1350F can be configured to break in the area of the second portion that is adjacent the gap defined between the first portion and the patch assembly 1302.

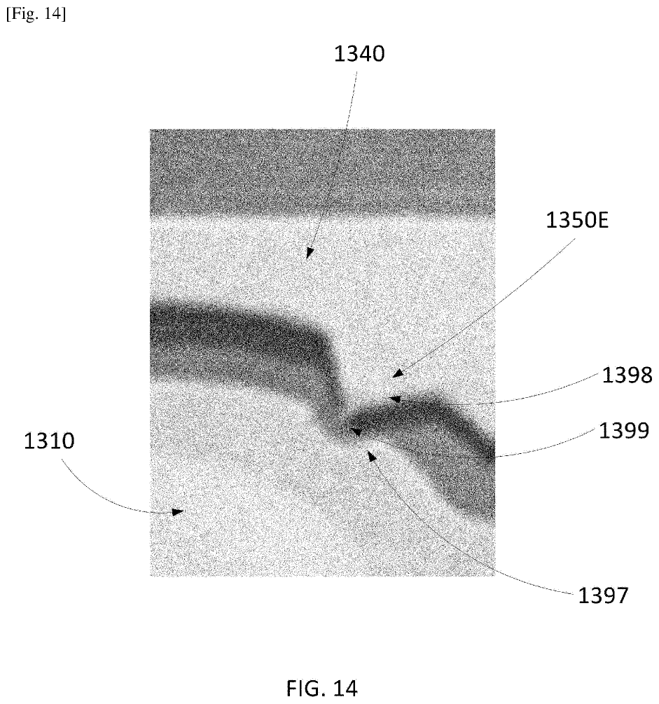

For example, FIG. 14 is a close-up view of a portion of FIG. 13 including the connector 1350E. As shown in FIG. 14, connectors 1350E includes a first portion 1398 (e.g., a top layer) including a first material and a second portion 1399 (e.g., a bottom layer) including a second material. The first portion 1398 is disposed on top of the second portion 1399, and extends only a portion of a distance between the frame assembly 1340 and the first portion 1310 of the patch assembly 1302. The first portion 1398 is tapered toward the first portion 1310 of the patch assembly 1302. The second portion 1398 extends the entire distance between the frame assembly 1340 and the first portion 1310 of the patch assembly 1302. As shown in FIG. 14, the first portion 1310 of the patch assembly 1302 can define a cut-out portion 1397 such that a gap is defined between the first portion 1398 and the first portion 1310 of the patch assembly 1302. The first portion 1310 of the patch assembly 1302 and the frame assembly 1340 can be coupled together by the second portion 1399 in the region of the cut-out portion 1397. The second portion 1399 can be configured to break in or near the region of the cut-out portion 1397 such that the frame assembly 1340 can be separated from the first portion 1310 of the patch assembly 1302. Each of the connectors 1350A-1350D and 1350F can be the same or similar in structure and/or function to the connector 1350E shown in FIG. 14.

Although the connectors 1350A-1350F of the system 1300 are shown as being triangular and tapered toward the patch assembly 1302, the systems described herein can include connectors having any suitable shape (e.g., tapered toward the frame assembly 1340, rectangular, etc.).

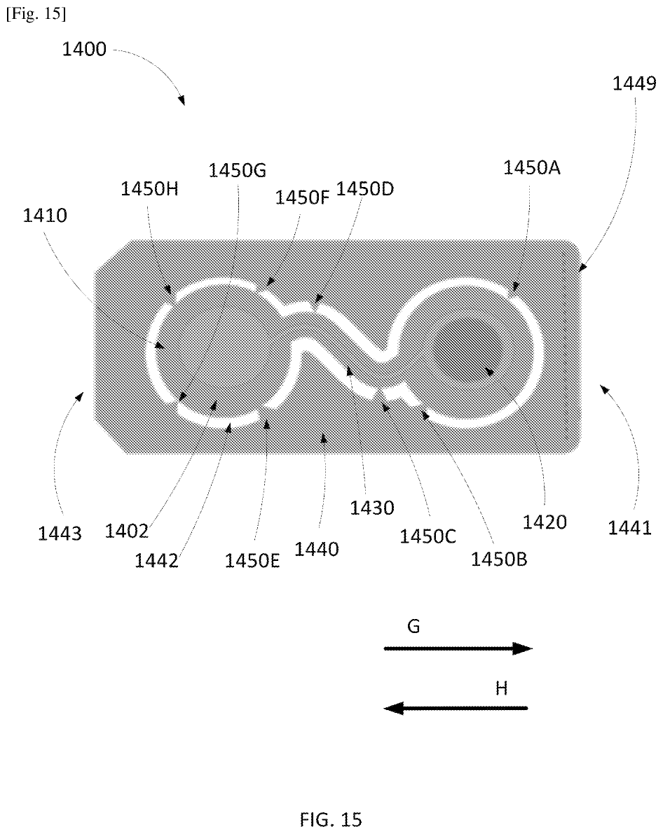

In some embodiments, a system can include a set of connectors that are disposed asymmetrically relative to a midpoint of a patch assembly of the system along a longitudinal axis of the patch assembly. For example, FIG. 15 is a top view of a system 1400 including a patch assembly 1402, a frame assembly 1440, and a protective layer (not shown). The system 1400 can be the same or similar in structure and/or function to any of the systems described herein. The protective layer can be coupled and/or bonded to a portion of the frame assembly 1440 via a hinge portion 1449. The hinge portion 1449 can include any suitable type of bonding such that, when a force (e.g., a pulling or peeling force) is applied to the protective layer to separate the protective layer from the patch assembly 1402 and the frame assembly 1440, the protective layer is separated from the patch assembly 1402 and a portion of the frame assembly 1440, but remains coupled to a portion (e.g., the hinge portion 1449) of the frame assembly 1440 (e.g., the first end 1441 of the frame assembly 1440). For example, the protective layer can be coupled to the patch assembly 1402 via a removable adhesive and to a portion of the frame assembly 1440 (e.g., the first end 1441 of the frame assembly 1440) via a non-removable adhesive at the hinge portion 1449.

The patch assembly 1402 is in a first patch configuration and is coupled to the frame assembly 1440 via eight connectors 1450A-1450H. Specifically, the patch assembly 1402 is coupled to the frame assembly 1440 via a first connector 1450A, a second connector 1450B, a third connector 1450C, a fourth connector 1450D, a fifth connector 1450E, a sixth connector 1450F, a seventh connector 1450G, and an eighth connector 1450H. Although the system 1400 is shown as including six connectors, in some embodiments the system can include any suitable number of connectors.

The patch assembly 1402 can be the same or similar in structure and/or function to any of the systems described herein. For example, the patch assembly 1402 includes a first assembly 1410, a second assembly 1420, and a connecting member 1430. As shown in FIG. 15, connectors 1450A-1450H can couple each of the first assembly 1410, the second assembly 1420, and the connecting member 1430 to the frame assembly 1440. Specifically, the first connector 1450A and the second connector 1450B can couple the second assembly 1420 to the frame assembly 1440. The third connector 1450C and the fourth connector 1450D can couple the connecting member 1430 to the frame assembly 1440. The fifth connector 1450E, the sixth connector 1450F, the seventh connector 1450G, and the eighth connector 1450H can couple the first assembly 1410 to the frame assembly 1440. Thus, more of the connectors 1450A-1450H can be disposed to couple the first assembly 1410 to the frame assembly 1440 than are disposed to couple the second assembly 1420 to the frame assembly 1440. Additionally, the connectors 1450A-1450H are disposed asymmetrically relative to a midpoint of the patch assembly 1402 along a longitudinal axis of the patch assembly 1402, with more of the connectors 1450A-1450H disposed on a side of the midpoint farther from the hinge portion 1449 than are disposed on the side closest to the hinge portion 1449. As a result, less shear force can be used to separate a first portion (e.g., the first half) of the patch assembly 1402 (e.g., the second assembly 1420 and a portion of the connecting member 1430) than is needed to separate a second portion (e.g., the second half) of the patch assembly 1402 (e.g., the first assembly 1410 and a remaining portion of the connecting member 1430).

As shown in FIG. 15, the connecting member 1430 has a first sinusoidal shape having a first frequency in the first configuration of the patch assembly 1402. The connecting member 1430 can also have a second sinusoidal shape having a second frequency in a second configuration of the patch assembly 1402 (not shown in FIG. 15). The second frequency can be different from the first frequency. For example, the connecting member 1430 may be shorter or longer in overall length in the second frequency compared to the first configuration. The opening 1442 defined by the frame assembly 1440 can include an opening portion having a sinusoidal shape having the first frequency such that the connecting member 1430 of the patch assembly 1402 can be disposed within the opening 1442 when the patch assembly 1402 is in the first configuration.

The frame assembly 1440 has a first end 1441 and a second end 1443. In use, prior to disposing the frame assembly 1440 and the patch assembly 1402 on the surface, the protective layer can be pulled away from the frame assembly 1440 and the patch assembly 1402 in the direction of arrow G such that the protective layer does not contact the patch assembly 1402 but remains coupled to the first end 1441 of the frame assembly 1440 at the hinge portion 1449. The system 1400 can be disposed on a surface (e.g., skin) of a user such that the patch assembly 1402 is in the first patch configuration and coupled to the surface via an adhesive portion (not shown). A user can grip the frame assembly 1440 to apply the system 1400 to the surface of the user without touching an adhesive portion of the patch assembly 1402. The frame assembly 1440 can then be separated from the patch assembly 1402 via pulling the first end 1441 of the frame assembly 1440 in the direction of arrow H (e.g., toward the second end 1443 of the frame assembly 1440) such that each of the connectors 1450A-1450H breaks (e.g., due to a shear force) and the frame assembly 1440 is separated from the patch assembly 1402. A portion of the frame assembly 1440 (e.g., an end such as the first end 1441 and/or the hinge portion 1449) can function as a handle during the separation of the frame assembly 1440 from the patch assembly 1402. The patch assembly 1402 can remain disposed on the surface after the frame assembly 1440 is separated from the patch assembly 1402. The patch assembly 1402, having been separated from the frame assembly 1440, can then transition between a first patch configuration and a second patch configuration to accommodate skin deformations of the user and reduce stress at the skin-adhesive interface.

Although the connectors 1450A-1450H of the system 1400 are shown as being triangular and tapered toward the patch assembly 1402, the systems described herein can include connectors having any suitable shape (e.g., tapered toward the frame assembly 1440, rectangular, etc.). Additionally, each of the connectors can be the same or similar in structure and/or function to any of the connectors described herein with respect to their composition and/or location relative to the patch assembly 1402 and/or the frame assembly 1440. In some embodiments, alternatively or additionally to the connectors 1450A-1450H being disposed asymmetrically about the midpoint of the patch assembly 1402, a subset of the connectors 1450A-1450H can be of a different shape, size, and/or material such that the subset is easier to break (e.g., requires less shear force) to separate the patch assembly 1402 from the frame assembly 1440. In some embodiments, the system 1400 does not include a protective layer and the end 1441 can be used as a handle to separate the frame assembly 1440 from the patch assembly 1402.

While various embodiments of the invention have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

In some embodiments, the systems (or any of its components) described herein can include a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices.

Although various embodiments have been described as having particular features and/or combinations of components, other embodiments are possible having a combination of any features and/or components from any of the embodiments where appropriate.

In some embodiments, a system includes a patch assembly and a frame. The patch assembly includes an adhesive portion and is configured to be coupled to a surface of a user via the adhesive portion. The patch assembly has a first patch configuration and a second patch configuration. The frame defines an opening. The patch assembly is configured to be disposed within the opening. The frame has a first frame configuration in which the frame is coupled to the patch assembly via a plurality of connectors such that the frame prevents the patch assembly from transitioning between the first patch configuration and the second patch configuration. The frame has a second frame configuration in which the plurality of connectors are broken and the frame is separated from the patch assembly.

In some embodiments, the adhesive portion is a first adhesive portion. The patch assembly includes a first assembly including the first adhesive portion, a second assembly including a second adhesive portion, and a connecting member coupled to the first assembly and the second assembly. The connecting member is configured to transition between a first configuration and a second configuration. The connecting member is in the first configuration when the patch assembly is in the first patch configuration and in the second configuration when the patch assembly is in the second patch configuration. A distance between the first end and the second end of the connecting member in the first configuration is a first distance. A distance between the first end and the second end of the connecting member in the second configuration is a second distance different from the first distance. The patch assembly is configured to be disposed within the opening and coupled to the frame via the plurality of connectors in the first configuration. The patch assembly is prevented from transitioning to the second configuration by the connection between the plurality of connectors and the frame.

In some embodiments, the connecting member is biased toward the first configuration.

In some embodiments, the patch assembly includes a first assembly including a first electrode and a second assembly including a second electrode.

In some embodiments, each connector from the plurality of connectors extends non-parallel to a longitudinal axis of the patch assembly.

In some embodiments, the frame is inelastic in a direction along a longitudinal axis of the patch assembly.

In some embodiments, each connector from the plurality of connectors is arranged such that each laterally-extending plane disposed perpendicular to a longitudinal axis of the patch assembly includes either a single connector or no connector.

In some embodiments, a system further comprises a protective layer removably coupled to a skin-facing surface of the patch assembly and non-removably coupled to a portion of a skin-facing surface of the frame.

In some embodiments, each connector from the plurality of connectors includes a first end coupled to the frame and a second end coupled to the patch assembly, each connector tapering from the first end to the second end.

In some embodiments, the shape of the opening defined by the frame corresponds to the shape of the patch assembly.

In some embodiments, the connecting member includes a first segment coupled to a second segment via a flexible hinge. The first segment and the second segment is arranged at a first angle when the connecting member is in the first configuration. The opening includes a first opening portion configured to receive the first segment and a second opening portion configured to receive the second segment. The first opening portion is disposed at a second angle relative to the second opening portion. The second angle is the same as the first angle.

In some embodiments, the connecting member has a first sinusoidal shape having a first frequency in the first configuration and a second sinusoidal shape having a second frequency in the second configuration. The second frequency is different from the first frequency. The opening in the frame includes an opening portion having a sinusoidal shape having the first frequency.

In some embodiments, a system includes a patch assembly, a frame assembly, and a protective layer. The patch assembly includes an adhesive portion configured to couple the patch assembly to a surface of a user. The frame assembly defines an opening. The frame assembly includes a top layer, an adhesive layer, and a liner layer. The adhesive layer is disposed between the top layer and the liner layer. The patch assembly is configured to be disposed within the opening and coupled to the frame via a plurality of connectors extending between the frame and the patch. The frame is configured to be removed from the patch assembly by breaking each connector from the plurality of connectors in a sequential manner. The protective layer is coupled to the adhesive portion and disposed in contact with a bottom surface of the liner layer.

In some embodiments, the adhesive portion is a first adhesive portion. The patch assembly includes a first subassembly including the first adhesive portion, a second subassembly including a second adhesive portion, and a connecting member coupled to the first subassembly and the second subassembly.

In some embodiments, the connecting member includes a third adhesive portion, the third adhesive portion coupled to the protective layer.

In some embodiments, the first adhesive portion, the second adhesive portion, and the third adhesive portion are included in a continuous adhesive layer.

In some embodiments, the first adhesive portion, the second adhesive portion, the third adhesive portion, the adhesive layer of the frame, and a portion of each connector of the plurality of connectors are included in a continuous adhesive layer.

In some embodiments, the first subassembly includes a first electrode and the second subassembly includes a second electrode.

In some embodiments, the protective layer has a first end and a second end and the frame assembly has a first end and a second end. The first end of the protective layer is bonded to the first end of the frame.

In some embodiments, a system includes a patch assembly, a frame, and a protective layer. The patch assembly includes an adhesive portion configured to couple the patch assembly to a surface of a user. The patch assembly has an outer perimeter. The frame defines an opening. The patch assembly is configured to be disposed within the opening and coupled to the frame via a plurality of connectors extending between the frame and the outer perimeter of the patch. A larger portion of the outer perimeter of the patch is free from the plurality of connectors than a total portion of the outer perimeter of the patch coupled to a connector from the plurality of connectors. The protective layer is coupled to the adhesive portion and in direct contact with a bottom surface of the frame.

In some embodiments, the adhesive portion is a first adhesive portion. The patch assembly includes a first subassembly including the first adhesive portion, a second subassembly including a second adhesive portion, and a connecting member coupled to the first subassembly and the second subassembly.

In some embodiments, a method includes separating a portion of a protective layer from a bottom surface of a patch assembly such that the protective layer is decoupled from an adhesive portion of the patch assembly. The patch assembly is disposed within an opening defined by a frame. The patch assembly is coupled to the frame via a plurality of connectors extending between the frame and the patch. The patch assembly and the frame are disposed on a surface of a user such that the adhesive portion couples the patch assembly to the surface. Each of the connectors from the plurality of connectors is decoupled such that the patch assembly remains coupled to the surface and the frame is removed from the surface.

In some embodiments, the portion of the protective layer from the bottom surface of the patch assembly is separated such that the protective layer remains coupled to the frame.

In some embodiments, decoupling each of the connectors of the plurality of connectors includes pulling a first end of the frame toward a second end of the frame such that each connector from the plurality of connectors breaks in series.

1. A system, comprising:
a patch assembly including an adhesive portion, the patch assembly configured to be coupled to a surface of a user via the adhesive portion, the patch assembly having a first patch configuration and a second patch configuration; and
a frame defining an opening, the patch assembly configured to be disposed within the opening, the frame having a first frame configuration in which the frame is coupled to the patch assembly via a plurality of connectors such that the frame prevents the patch assembly from transitioning between the first patch configuration and the second patch configuration, the frame having a second frame configuration in which the plurality of connectors are broken and the frame is separated from the patch assembly.

2. The system of claim 1, wherein the adhesive portion is a first adhesive portion, the patch assembly including a first assembly including the first adhesive portion, a second assembly including a second adhesive portion, and a connecting member coupled to the first assembly and the second assembly, the connecting member configured to transition between a first configuration and a second configuration, the connecting member being in the first configuration when the patch assembly is in the first patch configuration and in the second configuration when the patch assembly is in the second patch configuration, a distance between the first end and the second end of the connecting member in the first configuration being a first distance, a distance between the first end and the second end of the connecting member in the second configuration being a second distance different from the first distance, the patch assembly configured to be disposed within the opening and coupled to the frame via the plurality of connectors in the first configuration, the patch assembly prevented from transitioning to the second configuration by the connection between the plurality of connectors and the frame.

3. The system of claim 2, wherein the connecting member is biased toward the first configuration.

4. The system of claim 1, wherein the patch assembly includes a first assembly including a first electrode and a second assembly including a second electrode.

5. The system of claim 1, wherein each connector from the plurality of connectors extends non-parallel to a longitudinal axis of the patch assembly.

6. The system of claim 1, wherein the frame is inelastic in a direction along a longitudinal axis of the patch assembly.

7. The system of claim 1, wherein each connector from the plurality of connectors is arranged such that each laterally-extending plane disposed perpendicular to a longitudinal axis of the patch assembly includes either a single connector or no connector.

8. The system of claim 1, further comprising a protective layer removably coupled to a skin-facing surface of the patch assembly and non-removably coupled to a portion of a skin-facing surface of the frame.

9. The system of claim 1, wherein each connector from the plurality of connectors includes a first end coupled to the frame and a second end coupled to the patch assembly, each connector tapering from the first end to the second end.

10. The system of claim 1, wherein the shape of the opening defined by the frame corresponds to the shape of the patch assembly.

11. The system of claim 2, wherein the connecting member includes a first segment coupled to a second segment via a flexible hinge, the first segment and the second segment arranged at a first angle when the connecting member is in the first configuration, the opening including a first opening portion configured to receive the first segment and a second opening portion configured to receive the second segment, the first opening portion disposed at a second angle relative to the second opening portion, the second angle being the same as the first angle.

12. The system of claim 2, wherein the connecting member has a first sinusoidal shape having a first frequency in the first configuration and a second sinusoidal shape having a second frequency in the second configuration, the second frequency different from the first frequency, and the opening of the frame includes an opening portion having a sinusoidal shape having the first frequency.

13. A system, comprising:
a patch assembly including an adhesive portion configured to couple the patch assembly to a surface of a user;
a frame assembly defining an opening, the frame assembly including a top layer, an adhesive layer, and a liner layer, the adhesive layer disposed between the top layer and the liner layer, the patch assembly configured to be disposed within the opening and coupled to the frame assembly via a plurality of connectors extending between the frame assembly and the patch assembly, the frame assembly configured to be removed from the patch assembly by breaking each connector from the plurality of connectors in a sequential manner; and
a protective layer coupled to the adhesive portion and disposed in contact with a bottom surface of the liner layer.

14. The system of claim 13, wherein the adhesive portion is a first adhesive portion, the patch assembly including a first subassembly including the first adhesive portion, a second subassembly including a second adhesive portion, and a connecting member coupled to the first subassembly and the second subassembly.

15. The system of claim 14, wherein the connecting member includes a third adhesive portion, the third adhesive portion coupled to the protective layer.

16. The system of claim 15, wherein the first adhesive portion, the second adhesive portion, and the third adhesive portion are included in a continuous adhesive layer.

17. The system of claim 15, wherein the first adhesive portion, the second adhesive portion, the third adhesive portion, the adhesive layer of the frame assembly, and a portion of each connector of the plurality of connectors are included in a continuous adhesive layer.

18. The system of claim 13, wherein the first subassembly includes a first electrode and the second subassembly includes a second electrode.

19. The system of claim 13, wherein the protective layer has a first end and a second end and the frame assembly has a first end and a second end, the first end of the protective layer bonded to the first end of the frame assembly.

20. A system, comprising:
a patch assembly including an adhesive portion configured to couple the patch assembly to a surface of a user, the patch assembly having an outer perimeter;
a frame assembly defining an opening, the patch assembly configured to be disposed within the opening and coupled to the frame assembly via a plurality of connectors extending between the frame assembly and the outer perimeter of the patch assembly, a larger portion of the outer perimeter of the patch assembly being free from the plurality of connectors than a total portion of the outer perimeter of the patch being coupled to a connector from the plurality of connectors; and
a protective layer coupled to the adhesive portion and in direct contact with a bottom surface of the frame assembly.

21. The system of claim 20, wherein the adhesive portion is a first adhesive portion, the patch assembly including a first subassembly including the first adhesive portion, a second subassembly including a second adhesive portion, and a connecting member coupled to the first subassembly and the second subassembly.

22. A method, comprising:
separating a portion of a protective layer from a bottom surface of a patch assembly such that the protective layer is decoupled from an adhesive portion of the patch assembly, the patch assembly disposed within an opening defined by a frame, the patch assembly coupled to the frame via a plurality of connectors extending between the frame and the patch assembly;
disposing the patch assembly and the frame on a surface of a user such that the adhesive portion couples the patch assembly to the surface; and
decoupling each of the connectors from the plurality of connectors such that the patch assembly remains coupled to the surface and the frame is removed from the surface.

23. The method of claim 22, wherein the portion of the protective layer from the bottom surface of the patch assembly is separated such that the protective layer remains coupled to the frame.

24. The method of claim 22, wherein the decoupling each of the connectors of the plurality of connectors includes pulling a first end of the frame toward a second end of the frame such that each connector from the plurality of connectors breaks in series.

25. A system, comprising:
(a) an electronic assembly having
a first sub-electronic portion having a first bottom side on which a first electrode is disposed;
a second sub-electronic portion having a second bottom side on which a second electrode is disposed; and
a third sub-electronic portion connecting the first sub-electronic portion and the second sub-electronic portion, the third sub-electronic portion having a third bottom side on which a conductive trace is disposed;
(b) a bottom adhesive layer removably provided on the first bottom side, the second bottom side, and the third bottom side of the electronic assembly, the bottom adhesive layer having
a first adhesive portion disposed on the first bottom side of the first sub-electronic portion, the first adhesive portion having a first opening such that the first electrode disposed on the first bottom side of the first sub-electronic portion is accessible through the first opening;
a second adhesive portion disposed on the second bottom side of the second sub-electronic portion, the second adhesive portion having a second opening such that the second electrode disposed on the second bottom side of the second sub-electronic portion is accessible through the second opening; and
a third adhesive portion disposed on the third bottom side of the third sub-electronic portion;
(c) a cover frame having
a first frame portion disposed on a first upper side of the first sub-electronic portion;
a second frame portion disposed on a second upper side of the second sub-electronic portion;
a third frame portion disposed on a third upper side of the third sub-electronic portion;
a surrounding frame portion surrounding the first, second, and third frame portions to form a surrounding opening extending along a periphery of the electronic assembly; and
a plurality of connectors, each connector from the plurality of connectors configured to connect at least one of the first frame portion, the second frame portion, or the third frame portion to the surrounding frame portion, wherein the surrounding frame portion of the cover frame is configured to be separated from the first frame portion, the second frame portion, and the third frame portion by breaking each connector from the plurality of connectors.

26. The system of claim 25, further comprising
(d) a protective layer removably disposed on a bottom side of the bottom adhesive layer.

27. The system of claim 25, wherein each of the third sub-electronic portion, the third adhesive portion, and the third frame portion extends sinusoidally.

28. The system of claim 25, wherein the first sub-electronic portion includes electronic components, the electric components being electrically connected to the first electrode and also electrically connected through the conductive trace on the third sub-electronic portion.

29. The system of claim 28, wherein the electronic components includes an integrated circuit.

30. The system of claim 28, wherein the second sub-electronic portion includes an energy storage, the energy storage being electrically connected to the electronic components of the first sub-electronic portion via the conductive wire.

31. The system of claim 25, wherein the system has a longitudinal axis along which the first sub-electronic portion and the second sub-electronic portion are positioned.

32. The system of claim 31, wherein the each of the connectors from the plurality of connectors extends non-parallel to the longitudinal axis of the system.

33. The system of claim 31, wherein some of the connectors from the plurality of connectors extend perpendicularly and other connectors from the plurality of connectors extend at an angle that is neither parallel nor perpendicular to the longitudinal axis of the system.

34. The system of claim 31, wherein the connectors from the plurality of connectors are disposed asymmetrically relative to the longitudinal axis.

35. The system of claim 31, wherein the surrounding frame portion is separated from the first frame portion, the second frame portion, and the third frame portion of the cover frame by pulling a peripheral end of the surrounding frame portion in a direction along the longitudinal axis while the plurality of connectors are broken in series.

36. The system of claim 25, wherein each connector from the plurality of the connectors has a triangular shape that tapers from the surrounding frame portion to at least one of the first frame portion, the second frame portion, or the third frame portion.

37. The system of claim 25, wherein the protective layer is non-removably fixed to a peripheral end portion of the surrounding frame portion of the cover frame.

38. The system of claim 37, wherein removing the protective layer from the bottom adhesive layer and then pulling the removed protective layer in a direction away from the peripheral end portion of the surrounding frame portion of the cover frame causes the plurality of connectors to be broken in series and, thereby, the surrounding portion of the cover frame to be separated from the first frame portion, the second frame portion, and the third frame portion of the cover frame.

39. A system, comprising:
(a) an electronic assembly having
a first sub-electronic portion having a first bottom side on which a first electrode is disposed;
a second sub-electronic portion having a second bottom side on which a second electrode is disposed; and
a third sub-electronic portion connecting the first sub-electronic portion and the second sub-electronic portion, the third sub-electronic portion having a third bottom side on which a conductive trace is disposed; and
(b) a bottom adhesive layer removably provided on the first bottom side, the second bottom side, and the third bottom side of the electronic assembly, the bottom adhesive layer having
a first adhesive portion disposed on the first bottom side of the first sub-electronic portion, the first adhesive portion having a first opening such that the first electrode disposed on the first bottom side of the first sub-electronic portion is accessible through the first opening;
a second adhesive portion disposed on the second bottom side of the second sub-electronic portion, the second adhesive portion having a second opening such that the second electrode disposed on the second bottom side of the second sub-electronic portion is accessible through the second opening; and
a third adhesive portion disposed on the third bottom side of the third sub-electronic portion, wherein the third adhesive portion is configured to extend along the third sub-electronic portion.

40. The system of claim 39, wherein the third sub-electronic portion and the third adhesive portion extend sinusoidally.
